(19) United States
(12) Patent Application Publication
(10) Pub. No.: US 2008/0145388 A1
(43) Pub. Date: Jun. 19, 2008

(54) Product for the Targeted Release of Two-Compartment Active Substances

(76) Inventors: Michael Roreger, Neuwied (DE); Qian-Yi Li, Pullheim (DE); Iris Schnitzler, Bonn (DE); Dierk Schumacher, Melsungen (DE)

Correspondence Address: Frommer Lawrence & Haug, 745 Fifth Avenue, New York, NY, 10151, US

(21) Appl. No.: 11/886,352

(22) PCT Filed: Feb. 16, 2006
(86) PCT No.: PCT/EP2006/001391

§ 371 Date: Sep. 14, 2007

(30) Foreign Application Priority Data
Mar. 15, 2005 (DE) 10 2005 012 247.7

(51) Int. Cl. A61K 8/02; A61K 9/70; A61K 9/10; A61Q 99/00

(52) U.S. Cl. 424/401; 424/402; 424/443; 424/484

(57) ABSTRACT

The invention relates to a product for the targeted release of active substances, especially detergents and/or cosmetic active substances. The inventive product has a layer structure, one layer being impermeable to the active substance and another layer being permeable to the active substance. The two layers define a compartment between them which contains the active substance and optionally a gas-releasing component. The product is preferably suitable for single use and can be used as a cosmetic agent and/or as a cleaning agent.

The invention relates to a product for the targeted release of active substances, in particular washing compositions and/or cosmetic active ingredients.

For cleaning objects, cosmetic active ingredients and washing compositions are supplied in the form of powders, granules, solutions, bars and lotions. In the case of manual cleaning with washing compositions in this form, the problem arises that the hands are exposed to the washing composition for a prolonged period and can be detrimentally affected by it. This can lead to undesired washing hands.

A further problem with using cosmetic active ingredients and washing compositions in such forms can lie in a poor doseability. Finally, washing compositions in particular, for example in the form of powders, can generally dissolve very rapidly in water which, when cleaning under running water (e.g. when showering) brings with it an increased requirement for washing compositions.

One approach to solving these problems consists in the use of cleaning wipes. U.S. Pat. No. 4,234,442 describes a sachet which consists of water-permeable material and two sachet zones of solid acid and alkali/soap constituent. A cleaning article based on a cellulose-containing nonwoven which is impregnated with surfactants and which comprises a gas-generating system of citric acid and sodium carbonate is described in U.S. Pat. No. 4,272,393. Cleaning articles impregnated with surfactants in sachet form are also known (U.S. Pat. No. 4,515,703, U.S. Pat. No. 4,600,620, U.S. Pat. No. 4,603,069 and U.S. Pat. No. 6,720,301). WO 97/43366 also describes one of this type, but for the specific application case of automatic washing machines. DE 197 45 964 discloses self-foaming instant shampoos based on a powder surfactant, sodium hydrogencarbonate and acid. The use of a PVA film for aqueous surfactants is reported in GB 2 118 61.

EP 1 102 577 describes a product in which cosmetic active substances are enclosed in solid powder form in a sachet made of nonwovens. By virtue of gas-generating substances, the active substances and active ingredients are transported in the presence of water via a water-permeable layer in the form of foam to the site of application. Uncontrolled escape of the active substance is not excluded in the case of this product; thus, for example when shaking, active substance can escape from the sachet. Furthermore, targeted release, targeted at the site of application, is not ensured, leading to possible undesired and/or uncontrolled skin contact with the active substance. The described product also additionally has the disadvantage that use of a powder as active substance can lead to agglomerations of the active substances and active ingredients in the sachet area and, as a result, areas can also arise where there is no active substance at all.

It is therefore an object of the invention to provide a product for the targeted release of active substances, in particular skincare and skin-cleaning active substances. The release should take place in a controlled manner, namely in such a way that the active substances are available specifically at the site of application and that, especially in the case of manual use, the hands are protected from excessive and uncontrolled contact with the active substance. Release of the active substance should also take place in a controlled manner over time, i.e. over a defined, preferably prolonged period. The escape of pulverulent active substances from the product should be avoided. The invention should make it possible to process as many different materials as possible so that the variability is as great as possible for the most diverse applications.

The object is achieved by a product which comprises at least one active substance and has a coated structure. The product comprises at least three layers, where a first layer (1) is essentially impermeable to the active substance and a further layer (3) is permeable to the active substance. Also present is a separating layer (2) which is located between layers (1) and (3). In the product, the presence of the separating layer (2) has the effect that there is a compartment (A) and a compartment (B) which are spatially separate from one another. Layer (1) and layer (3) enclose these two compartments (A) and (B) and the separating layer (2) and are firmly joined together at the protruding edge regions via the separating layer (2).

Compartment (B) contains the at least one active substance and compartment (A) contains at least one gas-releasing component and optionally further auxiliaries.

This construction and the material properties of the separating layer (2) enable the use of a large number of different materials of layers (1) and (3) with a very variable profile of properties. Furthermore, the product is capable of releasing the active substance in a controlled manner. Release of the active substance takes place in a targeted manner through the layer (3) permeable to the active substance. This preferred direction is assisted by the material composition of layer (3), the presence of separating layer (2) and by the preferred use of an essentially water-impermeable layer (1). Since it is at the same time ensured that the active substance does not escape through layer (1), particularly in the case of manual application, undesired contact between the active substance and the hands is effectively excluded.

In a preferred embodiment, the product has a fixing means (4) on the side of layer (1) which faces away from compartment (B). Using this fixing means (4), the product can be attached securely to a hand, especially during manual application. With the fixing means (4), it can also, if appropriate, be ensured that the product is not inadvertently used sideways during manual application. Preferably, the fixing means (4) can have the form of a simple hanger, a loop or a glove-like form. It can be produced, for example, from textile or film-like material and be attached to layer (1) by sticking, welding or stitching.

The product is preferably envisaged for a single use. It can be used as cosmetic agent and/or as cleaning agent. Specific fields of use are body care (for example body care of ill or frail people by appropriately trained care personnel), massage, peeling, the cleaning and care of dishes (manually or by machine), windows, vehicles, textiles and surfaces of all types. On account of the single use as intended, even heavily soiled objects can be cleaned since soil residues which may adhere to the product itself can be disposed of together with it. A preferred form of application consists in using the product in an aqueous medium.

Suitable materials for layer (1), which is essentially impermeable to the active substance, are film-forming or fiber-forming substances, which may be used in the form of films, fibers, nonwovens, wovens, knits or microfibers (=fibers with an average fineness=“titer” of from 0.1 to 0.3 dtex). These include materials such as cellulose, viscose, pulp, cotton, polylactate acetate, polyethylene, polyethylene terephthalate, polypropylene, polyamides, polytetrafluoroethylenes, polyesters and mixtures thereof. It is also possible to use materials which are known for the manufacture of rubber gloves. These materials include polymers such as silicones, natural and synthetic rubbers, polyacrylonitriles, polyisoprenes and other materials known to the person skilled in the art. Preferably, polyethylene and polypropylene films and nonwoven laminates are suitable. Particular preference is given to two- or three-layered materials of the Sawatex series from Sandler AG (Schwarzenbach/Saale), in which a fiber distribution gradient is possible. Spun-laced and spun nonwovens with barrier coating and polyethylene films (breathable and also nonbreathable) can also be used.

For the purposes of this description, the property “essentially impermeable” means primarily impermeability of layer (1) to the active substance. However, this “impermeability” does not necessarily have to mean 100% impermeability to the active substance, although this is preferred. Since the product is only used for a limited period and, moreover, the separating layer (2) and the contents of compartment (A) can exert a certain barrier effect toward the active substance, it suffices to choose the impermeability of layer (1) in such a way which effectively prevents an amount of the active substance from being able to escape during the application period from layer (1), which could bring about an undesired effect within this period.

However, layer (1) may also be impermeable to water, which is preferably realized by a film-like or laminate-like nature. The layer thickness of layer (1) is expediently less than 2 mm, preferably less than 500 μm. Layer (1) can particularly preferably have a layer thickness between 9 μm and 25 μm.

Layer (1) should preferably also be impermeable to gas—in particular CO₂, N₂ and/or O₂—so that the gas developed in compartment (A) can primarily escape from the product through the separating layer (2), compartment (B) and layer (3). Adequate gas impermeability is likewise preferably ensured by a film-like or laminate-like nature of layer (1).

In a further embodiment of the product, layer (1) can comprise fragrances, which are applied, for example, in the spray method.

Compartment (B) contains at least one active substance. Its thickness, i.e. the distance between separating layer (2) and layer (3) should not exceed 5 mm for the purpose of making the product easy to handle. However, since, during application, dissolution of the active substance and gas evolution take place, this distance can increase considerably especially during application.

The width and length of compartment (B) are not subject to a technical restriction. However, on account of the amount of active substance required for a single use, the length and the width of compartment (B) are generally at least 1 cm. Compartment (B) contains at least the amount of active substance required for a single use. Therefore, compartment (B)—in the state prior to application—in practice has a volume between about 5 cm³ and 300 cm³.

The active substances can be present as solid and/or as liquid in compartment (B). Preferably, they are in the solid aggregate state, which in the case of liquid active substances can be achieved, if appropriate, through absorption/adsorption on suitable carrier substances. The active substances can thus be in the form of a powder, granules, flakes, tablets, microencapsulated liquid or solids, CPF powders (“concentrated powder form”, i.e. as flowable powder with a liquid fraction of at least 10%, preferably at least 30%, on a pulverulent carrier. These CPF powders can be produced in accordance with WO 99/17868, to which reference is made in its entirety) and the like. In this form, the active substances preferably have a minimum particle size which is larger than the pore size of the materials of layer (3) that depends on the weave pattern, the thread density and/or the weight per unit area.

Preferably, however, the active substances are in the form of a sheet-like or belt-like matrix, which can assume a form, longitudinal extension and width extension adapted to compartment (B). Such a sheet-like or belt-like matrix preferably has a height (=distance between separating layer (2) and layer (3)) which does not exceed 5 mm. This matrix particularly preferably has a height between 100 μm and 2 mm.

If the active substances are present in compartment (B) in a sheet-like or belt-like matrix, the latter preferably also has means which facilitate an ingress of water. Such means include holes, pores, channels, etc. In a particular embodiment, the matrix containing the active substance can also contain air bubbles, as a result of which it can assume the properties of a disperse system.

As a result of the fact that the active substances are present in a sheet-like or belt-like matrix, compared to the particulate form (powder, granules, flakes, etc.), the risk that they can become enriched or decimated upon storage of the product in one section of compartment (B) is clearly reduced. Possible problems with regard to agglomeration and/or separation of the active substances and of any other constituents of compartment (B) which may be present are also avoided. As a consequence of using such an “active substance matrix”, the amount of active substance released during use, based on areal sections of layer (3), is very constant.

Compartment (B) can also contain at least one carrier substance which imparts increased strength to this layer. The use of such carrier substances is particularly useful if the active substance is liquid or readily volatile. The carrier substance can, through its presence, contribute to stronger attachment of the active substance in compartment (B). Suitable carrier substances are natural and synthetic polymers. Preference is given to polymers which also have film-forming or structure-forming properties. Of suitability for this purpose are polyamides, polyacrylates, polyaminoacids, polyvinyl acetate, polyvinyl alcohol, polyethylene glycols, polysaccharides, polyvinyl-pyrrolidones, pullulan, alginic acid, starch, polyols, pigments, mica, cellulose and cellulose derivatives. In a particular embodiment, the carrier substance is water-soluble. For this reason, polyvinyl acetate, polyvinyl alcohol, polyvinylpyrrolidone and cellulose derivatives that are soluble or at least swellable in water, in particular, are preferred. Auxiliaries known to the person skilled in the art may also be present in compartment (B).

Compartment (A) comprises a gas-releasing component. This is to be understood as meaning a substance or a mixture of substances which is capable of producing a gaseous substance upon contact with water. This component is in the form of a solid, preferably in the form of powder, granules or flakes. The gas-releasing component is used in particular in order to make contact with water during use and to produce a gas which, in cooperation with an active substance which may be present in compartment (B), to bring about or to enhance foam formation.

These components include carbonates and/or hydrogen-carbonates, but also peroxo compounds and azides and which, in the mixture with proton donors, release gaseous carbon dioxide (CO₂), oxygen (O₂) or nitrogen (N₂). In particular, alkali metal, alkaline earth metal and ammonium bicarbonates and hydrogencarbonates can be used. Suitable proton donors are inorganic and organic acids, such as citric acid, tartaric acid, malic acid, succinic acid, oxalic acid, boric acid or amidosulfuric acid, but also substances such as hydrogensulfates or dihydrogenphosphates.

Since this reaction of the carbonate or hydrogen-carbonate with proton donors only takes place in practice in aqueous solution and the components in the product are present in solid and dry form until the product is used, it is possible to control the release of the gaseous substance (carbon dioxide, oxygen, nitrogen) and thus the foam formation, which optionally takes place subsequently in cooperation with the washing agent in such a way that said events only occur when the product is used in aqueous medium.

Preferred peroxo compounds are potassium monopersulfate and sodium perborate, the while the azide used is preferably sodium azide. Preferred carbonates and hydrogencarbonates include Na₂CO₃, NaHCO₃, K₂CO₃, KHCO₃, (NH₄)₂CO₃; preferred proton donors include citric acid and KHSO₄. The carbonates and hydrogencarbonates and the proton donors are preferably used in equimolar amounts (i.e. two protons per CO₃²-ion) in order to achieve as complete a CO₂ release as possible.

In the cooperation of the released gas with the active substance—if this active substance is selected from the group of washing agents—foam formation takes place. The foam escapes from the product through the layer (3). This foam is preferably fine-pored. Here, the nature of the foam can be influenced by the properties of layer (3)—in particular its pore structure—and also to a certain extent by the properties of the separating layer (2).

Stabilizers for the gas-releasing component may also be incorporated into compartment (A). They can counteract possible clumping of the gas-releasing component. Stabilizers which can be used are various substances, preferably starch or corn starch.

Compartment (A) is preferably flat in order to minimize the risk of uneven distribution of the gas-releasing component present as powder or granules. “Flat” is to be regarded as a thickness (=distance between separating layer (2) and layer (1)) below 5 mm, preferably below 2 mm. This can be achieved, for example, by using materials which only have very low elasticity for layer (1) and separating layer (2). The volume of compartment, (A) also depends on the use purpose intended in each case and the associated “gas requirement”. Preference is given to volumes between 4 cm³ and 300 cm³.

In a preferred embodiment of the product, compartment (A) can, in addition to the gas-releasing component, also comprise fragrances which are preferably applied in the spray method to the gas-releasing component present as solid. In a further embodiment, compartment (A) can also comprise at least one of the foam boosters specified under the active substances.

Furthermore, the product comprises a separating layer (2). Besides separating compartments (A) and (B), this has the effect that the gas-releasing component present in compartment (A) cannot emerge therefrom in an uncontrolled way. The separating layer (2) also serves for dimension and film stabilization, for which reason it is preferably used in the form of a nonwoven, a microfiber (=fibers with an average fineness=“titer” of from 0.1 to 0.3 dtex) of a film and/or of a laminate. Suitable materials for this are cellulose, viscose, polyethylene, polyethylene terephthalate, polypropylene, polyester, polylactate acetate, cotton and mixtures thereof. Preference is given to using a very thin spun-bonded, needled or thermobonded nonwoven made of polypropylene. The presence of the separating layer (2) can also in practice lead to layers (1) and (3) possibly being better bonded together. This is the case particularly if these two layers consist of very different materials which may not be particularly easy to join together.

Suitable materials for layer (3) are film-forming or fiber-forming materials. These materials can be used in the form of a woven, a knit, a microfiber (=fibers with an average fineness=“titer” of from 0.1 to 0.3 dtex) or a nonwoven (spun-laced, spun-bonded and/or needled nonwovens). These materials include, inter alia, cellulose, cotton, viscose, pulp, polyethylene, polyethylene terephthalate, polypropylene, polyester, polylactate acetate and mixtures of these. Since these materials are in the form of a woven, a knit or a nonwoven, the layer (3) is water-permeable. Here, the water permeability can be influenced by the weave pattern, the fiber density and/or the weight per unit area.

Preference is given to using nonwoven laminates of two- and three-layered materials of the Sawatex series from Sandler. In this connection, as layer (3), it is possible to use a Sawatex laminate which has a fiber distribution or hydrophilicity gradient. The outer layer of such a laminate is more hydrophilic than the inner layer. This hydrophilicity gradient is achieved through a high content of a hydrophilic polymer (e.g. viscose) in the outer layer and a high content of a hydrophobic polymer (e.g. polyethylene, polypropylene or polyester) in the inner layer. Any middle layer present can be between the two other layers with regard to its hydrophilic or hydrophobic character. This fiber distribution or hydrophilicity gradient—in particular the use of hydrophilic constituents in the outer layer of such a laminate—can additionally assist the targeted release of the active substance present in compartment (B).

For layer (3), particular preference is given to the use of cellulose, viscose and pulp and mixtures of these materials. The use of a padded nonwoven—for example in combination with a further laminate or nonwoven which can impart the required strength in this composite of layer (3)—is also advantageous because this brings about a padded feel when used on the skin.

In a further preferred embodiment, layer (3) can have abrasive properties. These can be produced through the presence of largely water-insoluble very finely divided powders (abrasives, cleaning bodies, polishes). Preferably, in such a case, a nonwoven is used which has strongly abrasive properties. A product equipped in such a way is used in the area of massage and peeling or domestically for cleaning hard surfaces.

On account of the water permeability of layer (3), water can enter the product and also exit again through this layer. The water which enters can dissolve the active substance present in compartment (B). The water can also effect the generation of gas from the gas-releasing component in compartment (A). Layer (3) is preferably also gas-permeable.

Layer (1) and layer (3) are preferably congruent. Like the separating layer (2), they are preferably larger than compartment (B) containing the active substance and compartment (A) containing the gas-releasing component. The two layers (1) and (3) and the separating layer (2) thus protrude when compartments (A) and (B) are laid over one another at the sides. There is then direct contact between the layers (1), (2) and (3) at these edge regions protruding at the side. In this area, these layers are firmly joined together, for example by sticking, ultrasound welding, hot melting, yarn stitching or other methods known to the person skilled in the art.

In a further embodiment of the product, the layer (3) can comprise fragrances.

Suitable active substances are, in particular, cosmetic active ingredients and/or washing agents. Cosmetic active ingredients are known to the person skilled in the art from the international guideline INCI (International Nomenclature Cosmetic Ingredients). The cosmetic active ingredients include skin protectants, skincare agents, skin oils and pharmaceutical active ingredients which act topically—i.e. in the epidermis. (Pharmaceutical active ingredients of this type can penetrate as far as the stratum corneum when applied to the skin, but do not enter the blood vessels.) For the purposes of this description, refatting agents, fragrances, foam boosters, glycerol, polyols, matting agents, stabilizers, antioxidants, dyes, antimicrobial additives, exfoliants and disinfectants can be counted as cosmetic active ingredients. In one particular embodiment, the product—in particular compartment (B)—can be free from preservatives and/or antimicrobial additives according to KVO.

The skin protectants include Abil Wax 9809, N-acylamino acid salts, Ajicoat SPQ, aluminum hydroxide, casein, Ceresperse Water Dispersible Waxes, Dermol, Dermolan L neutral, Eucornol, Finebase, Skin Protectant O-48-G, Lauridite, linoleic acid (dimerized), perfluoropolyether, polyvinyl alcohol, polyvinylpyrrolidonetriacontene polymer, Praestabitol V, Quick Break, Revitalin, Rewoderm S 1330, Sebosan S, starch ester, stearyl heptanoate and styrene-maleic acid copolymer.

The skincare agents include Abil WE-09, Alcolose W 2, Allantoin, Arosulf CL-A1, Bibranol, Biocorno, bisdiglyceryl ether, cholesterol ether, cholesterol polyglycol ether, cholesterol-siloxane compounds, cholesteryl oleate, Choleth, Chrestalan, Clearcol, coconut fatty acid 2-ethylhexyl, ester, Collapuron DAK, Condipon, decaglyceryl monooleate monosuccinate, dextran fatty acid ester, Diacetin, dicyclohexyalkanes, 1,5-dimethyl-2-isopentylhexanol fatty acid esters, dioctyl maleate, Dow Corning 225C, egg oil, Epiderma-sterols, Epigan, Epikuron, Estalan, ethyl avocadate, fatty acid dextrin ester, fatty acid diester, Fitoderm, Fluid E-370, Fomblin, Gafquat, Gluadin, glyceryl 3,5,3-trimethylhexanolate, guanidine, urea-D-glucoronic acid condensate, cis-6-hexadecenoic acid, hexaglycerol distearate tetraacetate, hexaglycerol hexastearate diacetate, 2,6,10,15,19,23-hexamethyltetracosane, Hexamol G-810, bis(2-hexyldecyltartrate), Hydagen P, Hydrocell YP-30, Hydrotriticum QM, hydroxyethyl-cellulose, Isodragol, lauryl isostearate, jojoba butter, Jordaquat JO-50, cocoa fruit juice, carrot oil, Katsernol, Kemester, levulinic acid, Lanacid, Lanesta, Lanoil, Lanolina C 500, Lantrol 1673, lecithin products, Lipocutin, LipoHyParts, liposaminic acids, Liposols, Lipotrofina A, Luteofilla, Menhaden oil, Mesil, methylheptadecanoic acid, Monaquat, 2-octyldodecyl myristate, Naetex Q, Natipide II, sodium lactate methylsilanol, sodium lauryl glutamate, sodium stearyl 2-lactylate, Necon DLD, Nerzolane, 9-octadecenyl octadecanoate, octadecyl vinyl ether, oleyl 2-hydroxypropionate, oleylpalmitylpalmitolamidopropyl derivatives, Phosal, Phospholipid EFA, Phospholipon, polyamino sugar condensate, polybutene, polydecenes, Polymer 28-4979, polymethacrylamidopropyltrimethylammonium chloride, polyquaternium-n, polyvinylpyrrolidone, Prolaurin, L-pyroglutamic acid, Quatrisoft LM-200, Sebopessina, Secol, silk amino acids, silk fibroin, sericin, silicone fatty acid ester, siloxane copolymers, soya sterols, sorbitol sulfate, Super Sterol Ester, stearic acid dimethylammonium chloride, Stearone, Surfactol Q series, tetrabutoxypropylmethicone, peat wax, Trifat S-308, Turtle Oil-R-Trixene, Usnagran, Visonoil-R and Wickenol 535 Vita Cos.

The skin oils include Cevenyl, Calendula Oil CLR, Cetiol, Cosmetic Liquid, Cosmetic natural oil, Cosmetol, Crodamol, Fluilan, Cyclal, di-2-hexyl tartrate, diisopropylidene triglycerol monostearate, 11,14-dioctyltetracosane, ethyl oleate, Fractionated Coconut Oil BP, rosehip seed oil, isodecane, isodecanoic acid ester, isohexaoctacontane, Isopar, javanicus oil, jojoba oil, Joleo, cherry stone oil, Kristole, kukui nut oil, ethyl linoleate, Liquid Base, Liquilan, Luvitol EHO, Mazula, Miglyol, Myritol 318, mink amidopropyldimethylamine acetate, mink oil fatty acid ethyl ester, mink oil polyethylene glycol ester, Nonanol, 2-ethylhexyl nonanoate, octyl neopentanoate, octyl octanoate, octyl pelargonate, olive oil fatty acid ester, Panalane L-14A, Patlac IL, plant oil CLR, polyethylene glycol (7) glyceryl cocoate, polyisoprene, Prisorine, Porbutyl, rice oils, Reisogran, silicone oils, sperm oil (substitute products), Super Refined Olive Oil, Tegosoft oils and Triisononanoin.

The refatting agents include higher fatty alcohols, higher fatty acids, triglycerides (fats), synthetic esters (isopropyl myristate, isopropyl palmitate or isopropyl adipate), wool wax derivatives and other substances.

The fragrances include single defined chemical compounds with odor and/or flavor. They are also termed odorants or osmogenes. Systematic arrangement of the fragrances is not in accordance with chemical structural features, but according to odor characteristics. They are arranged according to scent families and according to characteristic scent notes. Apart from the manufacture of perfumes, fragrances have diverse uses for the perfuming of soaps, deodorants, hair treatment compositions and other body care compositions, of detergents and cleaners, household articles, as odor improvers in technical products, in room air fresheners and room sprays, in the food and luxury product industry as aromas, essences and spice constituents (food additives). Fragrances are known to the person skilled in the art from the Code of the International Fragrance association (IFRA). Essential oils are also types of fragrances.

The foam boosters include interface-active substances which are added in small amounts in order to counteract rapid foam disintegration. Preference is given to using sodium lauryl sulfate, ammonium lauryl sulfate, triethanolamine lauryl sulfate, sodium dodecylbenzyl-sulfonate and/or sodium cocoyl isethionate.

The polyols include polyhydric alcohols which contain at least two alcoholic hydroxy groups in the molecule. These include diols, glycols, glycerol, etc., sugar alcohols, such as sorbitol and inositol, pentaerythritol, trimethylolpropane. The polyphenols are also types of polyols as are polyalkylene glycols, polyethylene glycols, polyether and polyester polyols.

The matting agents include substances such as cellulose nitrate, cellulose acetobutyrate, titanium dioxide, silicon dioxide, silk powder and talc.

The stabilizers include antiaging agents, light stabilizers, metal deactivators, ethylenediaminetetraacetic acid or magnesium silicate, but also preservatives, agents that prevent settling, dispersion auxiliaries, emulsifiers, foam stabilizers, etc.

The antioxidants include compounds of different types of chemical structure which suppress or prevent undesired changes caused by the effect of oxygen and other oxidative processes in the substances to be protected. Of suitability for this are natural substances (e.g. tocopherols, tocotrienols, flavonoids), but also synthetic substances, such as, for example, ascorbyl palmitate and gallic acid esters. Particular preference is given to propyl gallate, octyl gallate, dodecyl gallate, butylhydroxyanisole and butylhydroxytoluene.

The dyes which can be used in the product are those known to the person skilled in the art from Annex 3 of the Cosmetics Ordinance. Suitable antimicrobial additives, exfoliants and disinfectants are known to the person skilled in the art.

The washing agents include surfactants, detergent polymers (in particular inorganic polymeric builders and organic polymers with various types of functional groups), bleaching systems, detergent enzymes, optical brighteners (whiteners) and fabric softener active ingredients, and combinations thereof. Particularly preferred washing agents are the surfactants.

Surfactants are interface-active substances which bring about wetting and rewetting of the surface to be cleaned with the “wash liquor” and in so doing create the prerequisite for cleaning.

The surfactants used are anionic, cationic, nonionic and/or amphoteric detergents.

Suitable anionic detergents are sulfonated and sulfated alkyl, arylalkyl and alkylaryl compounds, alkyl succinates, alkyl sulfosuccinates and N-alkoyl sarcosinates. Preferences given to sodium, magnesium, ammonium and the mono-, di- and triethanolamine salts of alkyl and arylalkyl sulfates, and the corresponding salts of alkylarylsulfonates. The alkyl groups of the detergents generally have 12 to 21 carbon atoms and may be unsaturated, but preferably saturated. Alkyl ether sulfates which contain 1 to 10 ethylene oxide or propylene oxide units per molecule can also be used.

Typical suitable anionic detergents which can be used according to the invention are sodium lauryl sulfate, sodium lauryl ether sulfate, ammonium lauryl sulfate, triethanolamine lauryl sulfate, sodium (C₁₄₋₁₆)-olefin-sulfonates, sodium myristyl ether sulfate, ammonium lauryl ether sulfate, disodium lauryl sulfosuccinate, ammonium lauryl sulfosuccinate, sodium dodecylbenzyl-sulfonate, sodium cocoyl isethionate and sodium n-lauroylsarcosinate. Particularly preferred surfactants in the product according to the invention are sodium n-lauryl sulfate, monoisopropanol laureth sulfate and sodium n-lauryl ether sulfate, where the latter in particular are characterized by particular skincare and/or skin-protecting properties.

Suitable cationic detergents are monoquaternary or bisquaternary ammonium compounds which carry at least one long-chain aliphatic radical having 10 to 26 carbon atoms. This long-chain aliphatic radical can contain an ester bond or an amide bond. Preference is given to hexadecylmethylammonium chloride.

Nonionic detergents which can be used are condensation products of ethylene oxide or propylene oxide with a long-chain alcohol, a long-chain amine or a long-chain carboxylic acid. Here, the aliphatic carbon chain generally comprises 8 to 20 carbon atoms and can be condensed with 5 to 20 ethylene oxide or propylene oxide units. Nonionic detergents which can be used are also alkyl polyglycosides having 8 to 14 carbon atoms in the alkyl chain.

The amphoteric detergents used are primarily betaines which carry long alkyl groups. These include cocodimethylcarboxymethylbetaine, lauryldimethylcarboxymethylbetaine, lauryldimethyl-α-carboxyethylbetaine, cetyldimethylcarboxymethylbetaine, but also sulfobetaines, such as cocodimethylsulfodimethylbetaine and amido- and amidosulfobetaines. The carboxybetaines and the amidobetaines are particularly preferred. Specific examples thereof are cocoamidopropylbetaine, laurylamidopropylbetaine, myristylamidopropylbetaine and mixtures of said compounds.

The detergent polymers include zeolites (silicates) and polycarboxylates. These substances have an ion exchange capacity and serve to soften water by taking up calcium and magnesium ions.

Bleaching systems include substances which permit oxidative degradation of impurities. These include sodium percarbonate, sodium perborate, peroxyacetic acid and optionally bleach activators, such as N,N,N′,N′-tetraacetylethylenediamine (TAED) and p-nonanoyloxybenzenesulfonate.

The detergent enzymes include proteases, amylases, cellulases and lipases which serve to remove certain types of soiling.

Optical brighteners (whiteners) are organic substances which, in solution or on a substrate, absorb UV light and emit the majority of the absorbed energy again as blue fluorescent light between 400 and 500 nm. Such substances are known to the person skilled in the art.

Fabric softener active ingredients used are cationic interface-active compounds with two hydrophobic groups which are joined to a quaternized di-triethanolamine or an analogous compound via ester bonds. These substances are known to the person skilled in the art under the name ester quats.

The total content of the active substance in the product can be between 1 and 95% by weight, preferably between 15 and 85% by weight.

Release of the active ingredient is essentially controlled by the degradation of the product, by the dissolution behavior of the active ingredient present in compartment (B) and by the gas-release behavior in compartment (A).

Here, the order of the layers (1), (2) and (3) of compartments (A) and (B), and the special properties of these layers with regard to the water permeability ensure control with regard to the direction of the release of the active substance.

The nature of the active substance present in compartment (B) can influence its dissolution behavior in water and thus exert control with regard to release of the active substance over time. The active substance present in compartment (B) is in the solid aggregate state and can dissolve upon contact with water. The period which is required to dissolve the at least one active substance in an aqueous medium is termed the dissolution time. It is essentially dependent on the layer thickness, on the state (powder, granules, flakes, etc. or sheet-like or strip-like) and on the composition of the constituents present in compartment (B) (active substances, carrier substances, auxiliaries). If an active substance matrix in compartment (B) optionally comprises holes, pores, channels and/or air bubbles; these can considerably reduce the dissolution time. The dissolution time of the active substances present in compartment (B) can thus be in the range from a few seconds (i.e. less than 15 seconds) to a few minutes, i.e. 5 to 15 minutes. Mechanical stress (e.g. rubbing of the hands) can also further increase the rate of the dissolution process. DE 102 41 597, to the entire contents of which reference is made, discloses a layered soap preparation in which the dissolution behavior is influenced by air bubbles present therein. The dissolution process of the active substance present in compartment (B) can of course also slow down if the product, when in use, is not exposed to water uninterruptedly.

The product has no pregiven preferred form and can therefore be, for example, rectangular, square—optionally with rounded corners—round or oval and be present individually in the form of a stack or in the form of a roll. In this embodiment, it can be regarded as a cushion containing active substance. In rectangular form, it can preferably have a size of 4 cm×5 cm to 25 cm×30 cm, where 4 cm×5 cm to 8 cm×10 cm is preferred. In the circular form, diameters between 5 cm and 12 cm are preferred.

In a further embodiment, the product can have the external shape of a hand, meaning that—particularly in the case of the simultaneous presence of fixing means (4) having the external shape of a hand—it can be worn like a glove for the back of the hand.

For the manual use of a product having fixing means (4), compartment (B) containing the active substance can be on the side of the palm of the hand or on the side of the back of the hand. However, in every case, it is ensured that release of the active substance from layer (3) of the product takes place away from the hand.

Furthermore, the product can also be in the form of a complete glove. In this form, the product preferably has the size and the shape of a normal human hand of a child, teenager or an adult. In this embodiment, the product can be configured in such a way that it assumes the classic five-finger variant or the shape of a mitten. Variations in between are also conceivable, such as, for example, with and without thumbs or with cut-out holes to leave thumbs free.

The figures serve to illustrate the invention:

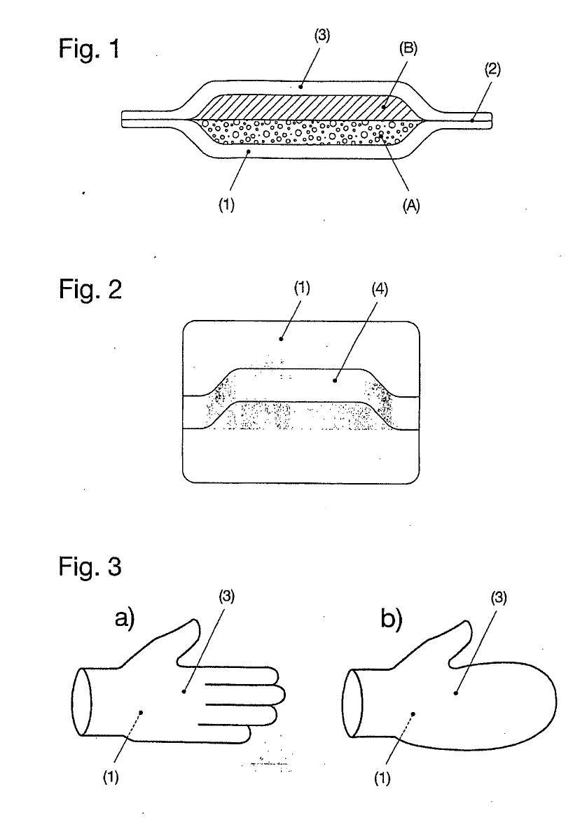

FIG. 1 shows the cross section of a product according to the invention with a layer (1), a separating layer (2) and a layer (3) which are firmly joined together at the protruding edge areas. Compartments (A) and (B) are separated from one another via the separating layer (2).

FIG. 2 shows, in top view, the layer (1) of a product which is equipped with fixing means (4) in the form of a loop made of a textile or film-like material.

FIG. 3 shows the product in the shape of a hand, in the five-finger variant (a) and as mitten (b).

The following examples also serve to illustrate the invention:

### EXAMPLE 1

### Production of a Rectangular Product

The composition of the active substances present in compartment (B) is shown in table 1 (initial weight 3.5 g). The formulation of the constituents of compartment (A) in which a gas-releasing component and fragrances and stabilizers are present is shown in table 2 (initial weight 13.5 g). The fragrances are sprayed onto the powder obtained using a spray tumble mixer. (In one variant, fragrances can be dispensed with. Its fraction is then put onto the other substances according to the distribution.) The size of the product is 8 cm×10 cm.

Layer (1) consists of a polypropylene film 5136591 (OPP Flow Wrap Film) from Nordenia, separating layer (2) consists of a polypropylene nonwoven with the name S1800PHW from Prato and layer (3) consists of a laminate Sawatex 2634 from Sandler.

TABLE 1

| Substance (INCI) | % by wt. |
|---|---|
| Sodium Lauryl Sulfate | 51.48 |
| Disodium Cocamido MEA Sulfosuccinate | 24.26 |
| Sodium Lauryl Glutamate | 24.26 |

TABLE 2

| Substance (INCI) | % by wt. |
|---|---|
| Citric Acid | 40.60 |
| Sodium hydrogencarbonate | 47.30 |
| Zea Mays | 10.10 |
| Perfume | 2.00 |

The finished product is produced by sticking or welding the various layers.

### EXAMPLE 2

As example 1, except that compartment (B) comprises the active substances given in table 3.

TABLE 3

| Substance (INCI) | % by wt. |
|---|---|
| Sodium Lauryl Sulfate | 51.44 |
| Laurylamidopropyl Betaine | 8.75 |
| Disodium Lauryl Sulfosuccinate | 24.23 |
| Dextrin | 9.10 |
| Sodium Hyaluronate | 0.45 |
| Sodium Lauryl Glutamate | 6.03 |

### EXAMPLE 3

As example 1, except that compartment (B) comprises the active substances given in table 4.

TABLE 4

| Substance (INCI) | % by wt. |
|---|---|
| Sodium Lauryl Sulfate | 51.52 |
| Laurylamidopropyl Betaine | 9.11 |
| Disodium Lauryl Sulfosuccinate | 21.20 |
| Dextrin | 6.04 |
| Sodium Hyaluronate | 3.02 |
| Rape seed oil | 9.11 |

### EXAMPLE 4

As example 2, except that in layer (3), the two-layered laminate Sawatex 2647 is used.

### EXAMPLE 5

As example 2, except that in layer (3) the laminate Sawatex 2652 is used.

### EXAMPLE 6

As example 2, except that in layer (3), the three-layered laminate Sawatex 2653 is used.

### EXAMPLE 7

As example 2, except that in layer (1), the two-layered laminate 05073FC28 and an LDPE film (breathable) is used.

### EXAMPLE 8

As example 2, except that in layer (1) the laminate 05023FC28 and an LDPE film (nonbreathable) is used.

### EXAMPLE 9

As example 2, except that layer (3) consists of a padded nonwoven 151-0060 from Lentex and the three-layered laminate Sawatex 2653.

### EXAMPLE 10

As example 2, except that layer (3) consists of the three-layered laminate Sawatex 2653 and an abrasive two-ply nonwoven SABD6SW480 from Shalag.

### EXAMPLE 11

As example 2, except that the product has a size measuring 10×15 cm.

### EXAMPLE 12

As example 2, except that the product has the shape of a human hand with five fingers. The underside of the hand consists here of a polypropylene film.

### EXAMPLE 13

As example 2, except that the product has the shape of a child's hand as a mitten. The underside of the hand here consists of a polypropylene film.

### EXAMPLE 14

The composition of the CPF powder present in compartment (B) is given in table 5 (initial weight 7.0 g). The formulation of compartment (A), in which substances for the release of gaseous substances, and also fragrances and stabilizers are present, is given in table 6 (initial weight 13.5 g). The fragrances are sprayed onto the powder obtained using a spray tumble mixer. (In one variant, fragrances can be dispensed with. The fraction is then added onto the other substances according to the distribution.) The size of the product is 8 cm×10 cm.

Layer (1) consists of a polypropylene film 5136591 (OPP Flow Wrap Film) from Nordenia, separating layer (2) consists of a polypropylene nonwoven with the name S1800PHW from Prato and layer (3) consists of a laminate Sawatex 2634 from Sandler.

TABLE 5

| Substance (INCI) | % by wt. |
|---|---|
| Silica | 50.00 |
| MIPA-Laureth Sulfate (and) Cocamido- | 17.98 |
| propyl Betaine | |
| PEG-7 Glyceryl Cocoate | 28.58 |
| Phenoxyethanol | 3.33 |
| 2-Bromo-2-nitropropane-1,3-diol | 0.11 |

TABLE 6

| Substance (INCI) | % by wt. |
|---|---|
| Citric Acid | 40.60 |
| Sodium hydrogencarbonate | 47.30 |
| Zea Mays | 10.10 |
| Perfume | 2.00 |

The finished product is produced by sticking or welding the various layers.

### EXAMPLE 15

As example 14, except that in layer (3) the three-layered laminate Sawatex 2653 is used.

### EXAMPLE 16

As example 15, except that layer (3) consists of a padded nonwoven 151-0060 from Lentex and the three-layered laminate Sawatex 2653.

### EXAMPLE 17

As example 15, except that layer (3) consists of the three-layered laminate Sawatex 2653 and an abrasive two-ply nonwoven SABD6SW480 from Shalag.

### EXAMPLE 18

As example 15, except that the product has the shape of a hand. The other side of the glove here consists of a polypropylene film.

### EXAMPLE 19

The composition of the active substances present in compartment (B) is shown in table 7. The fraction of active substance in compartment (B) is 15% by weight of the total content of compartment (B) (initial weight 3.5 g). The carrier substance is produced in accordance with the patent specification DE 102 41 597 B4 from SCS Skin Care Systems GmbH (initial weight 20.0 g). The formulation of the constituents of compartment (A), in which a gas-releasing component, and also fragrances and stabilizers are present, is given in table 8 (initial weight 13.5 g). The fragrances are sprayed onto the powder obtained using a spray tumble mixer. (In one variant, fragrances can be dispensed with. The fraction is then added onto the other substances according to the distribution.) The size of the product is 8 cm×10 cm.

Layer (1) consists of a polypropylene film 5136591 (OPP Flow Wrap Film) from Nordenia, separating layer (2) consists of a polypropylene nonwoven with the name S1800PHW from Prato and layer (3) consists of a laminate Sawatex 2634 from Sandler.

TABLE 7

| Substance (INCI) | % by wt. |
|---|---|
| MIPA-Laureth Sulfate (and) Cocamido- | 35.96 |
| propyl Betaine | |
| PEG-7 Glyceryl Cocoate | 57.15 |
| Phenoxyethanol | 6.67 |
| 2-Bromo-2-nitropropane-1,3-diol | 0.22 |

TABLE 8

| Substance (INCI) | % by wt. |
|---|---|
| Citric Acid | 40.60 |
| Sodium hydrogencarbonate | 47.30 |
| Zea Mays | 10.10 |
| Perfume | 2.00 |

The finished product is produced by sticking or welding the various layers.

### EXAMPLE 20

As example 19, except that in layer (3) the two-layered laminate Sawatex 2647 is used.

### EXAMPLE 21

As example 19, except that in layer (3) the laminate Sawatex 2652 is used.

### EXAMPLE 22

As example 19, except that in layer (3) the three-layered laminate Sawatex 2653 is used.

### EXAMPLE 23

As example 22, except that in layer (1) a polyethylene film is used.

### EXAMPLE 24

As example 22, except that in layer (1) a polyester film is used.

### EXAMPLE 25

As example 22, except that in layer (1) a polyethylene terephthalate film is used.

### EXAMPLE 26

As example 22, except that in layer (1) the two-layered laminate 05073FC28 and an LDPE film (breathable) is used.

### EXAMPLE 27

As example 22, except that in layer (1) the laminate 05023FC28 and an LDPE film (nonbreathable) is used.

### EXAMPLE 28

As example 19, except that the active substance content in compartment (B) is 45%.

### EXAMPLE 29

As example 19, except that the active substance content in compartment (B) is 55%.

### EXAMPLE 30

As example 19, except that the active substance content in compartment (B) is 65%.

### EXAMPLE 31

As example 22, except that compartment (B) comprises the active substances given in table 9.

TABLE 9

| Substance (INCI) | % by wt. |
|---|---|
| Sodium Lauryl Sulfate | 52.94 |
| Cocamide DEA | 23.53 |
| Laurylamidopropyl Betaine | 23.53 |

### EXAMPLE 32

As example 31, except that in layer (3) a spun-lace nonwoven made of polyester is used.

### EXAMPLE 33

As example 31, except that in layer (3) a spun-lace nonwoven made of polypropylene is used.

### EXAMPLE 34

As example 22, except that compartment (B) comprises the active substances given in table 10.

TABLE 10

| Substance (INCI) | % by wt. |
|---|---|
| Sodium Laureth Sulfate | 41.72 |
| Cocamidopropyl Betaine | 17.88 |
| PEG-7 Glyceryl Cocoate | 18.63 |
| Phenoxyethanol | 2.24 |
| Cocamide DEA | 18.63 |
| 2-Bromo-2-nitropropane-1,3-diol | 0.90 |
| 5-Chloro-2-methyl-4-isothiazolinone | |
| 2-Methyl-4-isothiazolin-3-one | |

### EXAMPLE 35

As example 22, except that compartment (B) comprises the active substances given in table 11.

TABLE 11

| Substance (INCI) | % by wt. |
|---|---|
| Sodium Laureth Sulfate | 88.30 |
| Cocamidopropyl Betaine | |
| Disodium Laureth Sulfosuccinate | |
| PEG-9 Cocoglycerides | |
| Decyl Oleate | 6.30 |
| Panthenol | 0.24 |
| Propylene glycol | 4.09 |
| Polyquaternium-6 | 0.44 |
| Phenoxyethanol, Ethylparaben, Methyl- | 0.63 |
| paraben, Butylparaben, Propylparaben | |

### EXAMPLE 36

As example 22, except that compartment (B) comprises the active substances given in table 12.

TABLE 12

| Substance (INCI) | % by wt. |
|---|---|
| Sodium Laureth Sulfate | 87.71 |
| Cocamidopropyl Betaine | |
| Disodium Laureth Sulfosuccinate | |
| PEG-9 Cocoglycerides | |
| Decyl Oleate | 6.50 |
| Tocopherol Acetate | 0.08 |
| Prunus Dulcis | 0.32 |
| Panthenol | 0.24 |
| Propylene glycol | 4.06 |
| Polyquaternium-6 | 0.45 |
| Phenoxyethanol, Ethylparaben, Methyl- | 0.64 |
| paraben, Butylparaben, Propylparaben | |

### EXAMPLE 37

As example 22, except that layer (3) consists of a padded nonwoven 151-0060 from Lentex and the three-layered laminate Sawatex 2653.

### EXAMPLE 38

As example 22, except that layer (3) consists of the three-layered laminate Sawatex 2653 and an abrasive two-ply nonwoven SABD6SW480 from Shalag.

### EXAMPLE 39

As example 19, except that the product has a size of 10×15 cm.

### EXAMPLE 40

As example 19, except that the product has the shape of a human hand with five fingers. The other side of the glove here consists of a polypropylene film.

### EXAMPLE 41

As example 19, except that the product has the shape of a child's hand as a mitten. The other side of the glove here consists of a polypropylene film.

1. A product for the controlled release of at least one active substance, comprising a layer (1) that is essentially impermeable to the active substance, a compartment (A) containing at least one active substance, and a layer (2) that is permeable to the active substance, characterized in that layer (2) comprises a nonwoven laminate which has a hydrophilicity gradient.

2. The product as claimed in claim 1, characterized in that the at least one active ingredient is a cosmetic active ingredient and/or a washing agent.

3. The product as claimed in claim 1, characterized in that it comprises, as active substance, at least one cosmetic active ingredient from the group of skin protectants, skincare agents, skin oils or pharmaceutical active ingredients which are effective in the epidermis.

4. The product as claimed in claim 1, characterized in that it comprises, as active substance, at least one washing agent from the group of surfactants, washing composition polymers, bleaching systems, washing composition enzymes, optical brighteners and softener active ingredients.

5. The product as claimed in claim 1, characterized in that layer (1) is in the form of a film, fiber, nonwoven, woven, microfiber or knit.

6. The product as claimed in claim 1, characterized in that layer (2) additionally comprises a padded nonwoven.

7. The product as claimed in claim 1, characterized in that layer (2) additionally comprises an abrasive material.

8. The product as claimed in claim 1, characterized in that layer (2) contains a material which is film-forming and/or fiber-forming and is selected from the group consisting of cellulose, viscose, cotton, pulp, polylactate acetate, polyethylene, polypropylene, polyethylene terephthalate, polyamide, polytetrafluoroethylene, polyester and mixtures thereof.

9. The product as claimed in claim 1, characterized in that the active substances in compartment (A) are in the form of a powder, granules, flakes, tablet, microencapsulated liquid, microencapsulated solid and/or as CPF powder.

10. The product as claimed in claim 1, characterized in that the active substances in compartment (A) are present in the form of a sheet-like or strip-like matrix.

11. The product as claimed in claim 1, characterized in that compartment (A) additionally comprises a gas-releasing component.

12. The product as claimed in claim 1, characterized in that the gas-releasing component is in the form of a powder, granules and/or flakes and is capable of producing a gaseous substance upon contact with water.

13. The product as claimed in claim 1, characterized in that the gas-releasing component is a mixture of carbonates and/or hydrogencarbonates with proton donors.

14. The product as claimed in claim 1, characterized in that it comprises a fixing means (3) on the layer (1).

15. The product as claimed in claim 1, characterized in that it has the outer shape of a glove, preferably as five-finger variant or as mitten.

16. The product as claimed in claim 1, characterized in that it has a rectangular, square, oval or round shape or combinations thereof.

17. The product as claimed in claim 1, characterized in that the active substance present in compartment (A) dissolves upon contact with water within a period of 15 minutes, preferably within 5 minutes.

18. The product as claimed in claim 1, characterized in that the active substance, following contact of the product with water, emerges from the layer (2) permeable to the active substance.

19. The product as claimed in claim 1, characterized in that the active substance, following contact of the product with water, is released over a period of up to 15 minutes.

20. A method for controlling the release of at least one active substance as cosmetic agent which comprises adding the product of claim to the at least one active substance.

21. A method for controlling the release of at least one active substance as cleaning agent which comprises adding the product of claim to the at least one active substance.
